(19) United States
(12) Patent Application Publication
(10) Pub. No.: US 2022/0233544 A1
(43) Pub. Date: Jul. 28, 2022

(54) TREATMENTS FOR SKIN CONDITIONS

(71) Applicant: ChemistryRX, Folcroft, PA (US)

(72) Inventor: Lars Brichta, Brooklyn, NY (US)

(21) Appl. No.: 17/587,380

(22) Filed: Jan. 28, 2022

Related U.S. Application Data
(60) Provisional application No. 63/199,848, filed on Jan. 28, 2021.

(51) Int. Cl. A61K 31/519; A61K 9/00

(52) U.S. Cl. CPC A61K 31/519; A61K 9/0014

(57) ABSTRACT

Compositions and methods for treating skin lesions using topically administered JAK/STAT inhibitors for treating skin lesions and disease such as bullous pemphigoid, bullous impetigo, bullous lichen planus, lichen planus of the mucosa, and the like.

### CROSS-REFERENCE TO RELATED APPLICATIONS

This application claims priority from U.S. Provisional No. 63/199,848, entitled “Treatments for Skin Conditions,” filed on Jan. 28, 2021, the entirety of which is hereby incorporated by reference.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

### BACKGROUND

Current methods, including invasive and non-invasive methods and formulations, are used to reduce the appearance of skin conditions, such epidermolysis bullosa simplex, congenital aplasia cutis, neonatal pemphigus, neonatal herpes gestationis, staphylococcal scalded skin syndrome, incontinentia pigmenti, epidermolytic ichthyosis, linear IgA dermatosis, bullous pemphigoid, bullous impetigo, bullous lichen planus, lichen planus of the mucosa, tuberous sclerosis-associated angiofibromas, angiofibromas, trichoepitheliomas, skin lesions associated with Birt-Hogg-Dube syndrome, of the skin of the face, skin lesions associated with Langerhans Cell Histiocytosis, Vascular malformations and tumors, Port Wine Stains, Kaposi sarcoma, Epidermal nevi, treatment-resistant hemangiomas, sensitive skin, fragile skin, or combinations thereof. Invasive techniques such as surgery, bulking agents (e.g., Restylane, Juvederm), laser resurfacing, produce more lasting effects for prominent defects. However, many consumers cannot afford or do not wish to undergo such aggressive cosmetic treatments.

Examples of non-invasive methods include concealing the defect by applying a foundation-type cosmetic to the skin or applying a cosmetic formulation that includes ingredients that can reduce the appearance of the defect over time. Unfortunately, foundations are not durable and do not reduce the appearance of significant skin imperfections, while cosmetic formulations containing ingredients that can reduce the appearance of imperfections take time to effect and also do not reduce the appearance of significant imperfections. The compositions and methods of embodiments described herein produce lasting improvements of significant skin imperfections without the need for surgery.

### SUMMARY OF THE INVENTION

Various embodiments are directed to methods for treating skin diseases, conditions, or disorders or symptoms thereof, including the step of topically administering to a subject in need of treatment a composition comprising up to about 5% (w/w) of a JAK/STAT inhibitor and a base. In some embodiments, the JAK/STAT inhibitor may be ruxolitinib (INCB 018424), tofacitinib (CP690550), AG490, momelotinib (CYT387), partcitinib (SB 1518), baricitinib (LY3009104), fedratinib (TG101348), BMS-911543, lestaurtinib (CEP- 701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, hyperforin, and pharmaceutically acceptable salts and combinations thereof. In certain embodiments, the JAK/STAT inhibitor may be tofacitinib.

The methods and compositions of the invention can be used to treat various skin conditions such as, for example, epidermolysis bullosa simplex, congenital aplasia cutis, neonatal pemphigus, neonatal herpes gestationis, staphylococcal scalded skin syndrome, incontinentia pigmenti, epidermolytic ichthyosis, linear IgA dermatosis, bullous pemphigoid, bullous impetigo, bullous lichen planus, lichen planus of the mucosa, tuberous sclerosis-associated angiofibromas, angiofibromas, trichoepitheliomas, skin lesions associated with Birt-Hogg-Dube syndrome, of the skin of the face, skin lesions associated with Langerhans Cell Histiocytosis, Vascular malformations and tumors, Port Wine Stains, Kaposi sarcoma, Epidermal nevi, treatment-resistant hemangiomas, sensitive skin, fragile skin, or combinations thereof.

### DESCRIPTION OF THE DRAWINGS

Not applicable

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 ml to 8 ml is stated, 2 ml, 3 ml, 4 ml, 5 ml, 6 ml, and 7 ml are also intended to be explicitly disclosed, as well as the range of values greater than or equal to 1 ml and the range of values less than or equal to 8 ml.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition, or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with the terms disease, condition, or illness, unless otherwise indicated.

The terms “effective amount” and “therapeutically effective amount” are used interchangeably in this disclosure and refer to an amount of a compound that, when administered to a subject, can reduce a symptom of a disorder in a subject or enhance, reduce, normalize, or adjust the growth, texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g. animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is an adult or child human.

The term “treating” is used herein, for instance, in reference to methods of treating a disorder or a condition, and generally includes the administration of a compound or composition which reduces the frequency of, or delays the onset of, symptoms of a medical condition or enhance, reduce, normalize or adjust the growth, texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition. For example, in the context of a bacterial, microbial, fungal, or protozoal infection, “treating” refers to the reduction in bacterial, microbial, fungal, or protozoal load and/or improvement in symptoms related to the infection.

As used herein, the term “therapeutic” means an agent utilized to treat, combat, ameliorate, prevent or improve an unwanted condition or disease of a subject. In part, embodiments described herein may be directed to the treatment of various skin diseases, conditions, or disorders or symptoms thereof, including, but not limited to, benign proliferations, neoplasms, superficial blood vessel anomalies (tumors and malformations), epidermolysis bullosa, wounds and sores, Langerhans Cell Histiocytosis, Tuberous sclerosis, premalignancies, or malignancies of the skin, as well as the enrichment of immune cells in the skin. The skin condition may be a virally induced or non-virally induced cutaneous growth or proliferation. The skin condition may be an inflammatory condition. The skin condition may be a hyperproliferative condition. The skin condition may be a genetically determined condition. The skin condition may be ageing including intrinsic and extrinsic changes (e.g., photoaging (ultraviolet light induced changes)), pigmentary changes, fine lines and rhytides. In some embodiments, the skin condition may be selected from Human Papilloma Virus induced lesions e.g., warts, common warts, palmoplantar warts, flat warts, recurrent warts, recalcitrant warts, treatment naïve warts, epidermodysplasia verruciformis related warts, anogenital warts, condyloma accuminatum, cervical dysplasias or neoplasias, e.g., cervical intraepithelial neoplasia (CIN); Herpesvirus related lesions including those induced by HHV-1 (HSV-1), HHV-2 (HSV-2), HHV-3 (varicella-zoster virus) e.g., chicken pox, Herpes zoster, shingles; Poxvirus induced lesions e.g., molluscum contagiosum, orf; callus, cutaneous horns, corns, acrochordons, fibroepithelial polyps, prurigo nodularis, actinic keratoses, squamous cell carcinoma, squamous cell carcinoma in situ, keratoacanthoma, basal cell carcinoma, cutaneous lymphomas and benign lymphocytic infiltrates & hyperplasias of the skin, clear cell acanthoma, large cell acanthoma, epidermolytic acanthoma, porokeratosis, hyperkeratosis, keratosis pilaris, lichenoid keratosis, acanthosis, acanthosis nigricans, confluent and reticulated papillomatosis, nevi, including e.g., dermal nevi, epidermal nevi, compound nevi, ILVEN (inflammatory linear verrucous epidermal nevi), nevus sebaceous, nevus comedonicus, and the like; acne, e.g., comedonal acne, inflammatory acne, papular acne, pustular acne, cystic acne; cysts, e.g., epidermoid cysts, milia, trichilemmal cysts, follicular cysts, proliferating cysts, dermoid cysts, pilonidal cysts, apocrine cysts, eccrine cysts, sebaceous cysts, mucous cysts, myxoid cysts, ganglion cysts, synovial cysts, vellus hair cysts, steatocystoma, hidrocystoma; adnexal neoplasms e.g., trichofolliculoma, fibrofolliculoma, perifollicular fibroma, trichodiscoma, nevus sebaceous, chondroid syringoma, trichoepithelioma, trichoblastoma, desmoplastic trichoepithelioma, pilomatricoma, pilomatrical carcinoma, tricholemmoma, trichelemmal carcinoma, tumor of the follicular infundibulum, tricoadenoma, proliferating pilar tumor, sebaceous hyperplasia, sebaceous adenoma, sebaceous epithelioma, sebaceous carcinoma, syringoma, poroma, hidradenoma, apocrine hidradenoma, spiradenoma, cylindroma, eccrine nevus (eccrine hamartoma), papillary adenoma, papillary adenocarcinoma; benign melanocytic proliferations or neoplasms e.g., ephilides, cafe-au-lait macules, Becker's melanosis, lentigines, solar lentigines, lentigo simplex, mucosal melanocytic lesions, Mongolian spots, Nevus of Ota, blue nevus, common acquired melanocytic nevi (nevocellular nevus, “moles”), congenital nevi, nevus spilus, recurrent nevi; vascular and perivascular neoplasms and reactive hyperplasias e.g., hemangiomas, cherry angiomas, hobnail hemangiomas (targeted hemosiderotic hemangiomas), tufted angiomas, hemangioendotheliomas, angiolymphoid hyperplasia with eosinophilia (ALHE), Glomus tumors (glomangiomas), hemangiopericytomas; cutaneous neural and neuroendocrine neoplasms e.g., neuromas, Schwannomas, neurofibromas, nerve sheath tumor, nerve sheath myxoma, neurothekeoma, granular cell tumor; fibrotic and fibrohistiocytic proliferations e.g., acrochordons, fibroepithelial polyps, fibromas, fibrous papules, angiofibromas, pearly penile papules, periungual fibromas, dermatofibromas, fibrokeratomas, sclerotic or pleomorphic fibromas, connective tissue nevi; cutaneous scars, hyperplasias, keloids, rosacea, cutaneous fungal, dermatophyte & mold infections, onychomycosis, hyperpigmentation, rhytides, psoriasis, malignant melanoma, seborrheic keratosis, seborrheic keratosis variants including e.g., dermatosis papulosis nigra, inverted follicular keratosis/keratoma warty dyskeratosis/warty dyskeratoma, acrokeratosis verruciformis, stucco keratosis; or a combination thereof.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to topical compositions containing one or more JAK/STAT inhibitors for treating skin lesions. In certain embodiments, the JAK/STAT inhibitor may be a tofacitinib. Other embodiments are directed to methods for treating skin lesions that include administering a topical composition containing one or more JAK/STAT inhibitors to a subject in need of treatment. The compositions of such embodiments may be formulated as topical compositions and may provide skin lesions healing, reduction in discoloration associated with skin lesions, and relief of symptoms associated with skin lesions.

The JAK/STAT inhibitors of such embodiments encompass all Jak/STAT inhibitors known in the art including any compound that inhibits expression or activity of Jakl, Jak2, Jak3, Tyk2, STAT1, STAT2, STAT3, STAT4, STAT5a, STAT5b, STATE, OSM, gp130, LIFR, OSM-Rp, protein or polypeptide. Such Jak/STAT inhibitors can be a protein, such as an antibody (monoclonal, polyclonal, humanized, chimeric, or fully human), or a binding fragment thereof. Antibody fragments can include, for example, single chain Fv (scFv), diabodies, Fv, and (Fab')2, triabodies, Fc, Fab, CDR1, CDR2, CDR3, combinations of CDR's, variable regions, tetrabodies, bifunctional hybrid antibodies, framework regions, constant regions, and the like and combinations thereof. Antibodies can be obtained commercially, custom generated, or synthesized against an antigen of interest according to methods established in the art. The Jak/STAT inhibitors of such embodiments include commercially available Jak/STAT inhibitors including, for example, ruxolitinib (INCB 018424), tofacitinib (CP690550), AG490, momelotinib (CYT387), partcitinib (SB 1518), baricitinib (LY3009104), fedratinib (TG101348), BMS-911543, lestaurtinib (CEP-701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, hyperforin, and the like and combinations thereof

In some embodiments, the Jak/STAT inhibitor may be a small molecule that binds to a protein and disrupts its function. Small molecules are a diverse group of synthetic and natural substances generally having low molecular weights. They can be isolated from natural sources (for example, plants, fungi, microbes and the like), are obtained commercially and/or available as libraries or collections. Candidate small molecules that modulate a protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries. In some embodiments, the agent is a small molecule that binds, interacts, or associates with a target protein or RNA. Such a small molecule can be an organic molecule that, when the target is an intracellular target, is capable of penetrating the lipid bilayer of a cell to interact with the target. Small molecules include, but are not limited to, toxins, chelating agents, metals, and metalloid compounds. Small molecules can be attached or conjugated to a targeting agent so as to specifically guide the small molecule to a particular cell.

The concentration of Jak/STAT inhibitor in such embodiments contain up to about 15% (w/w) JAK/STAT inhibitors. For example, in some embodiments, the composition may include about 0.25% (w/w) to about 15% (w/w), about 0.5% (w/w) to about 10% (w/w), about 0.75% (w/w) to about 7.5% (w/w), about 1% (w/w) to about 5% (w/w), about 1% (w/w) to about 3% (w/w), or any range or individual concentration of Jak/STAT inhibitor encompassed by these example ranges. In particular embodiments, the composition may include about 0.25% (w/w) to about 5% (w/w) ruxolitinib, tofacitinib, momelotinib, partcitinib, baricitinib, fedratinib, lestaurtinib, fludarabine, epigallocatechin-3-gallate (EGCG), momelotinib, pacritinib, peficitinib, decernmotinib, filgotinib, gandotinib, cerdulatinib, or combinations thereof, and in some embodiments, the compositions may include about 0.25% (w/w) to about 5% (w/w) tofacitinib or a derivative, including deuterated derivatives, thereof.

In certain embodiments, the compositions may include a base such as, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, and the like and combinations thereof.

In some embodiments, the base may be a liposomal base. Liposomal bases are an emulsion that includes a lipophilic component and an aqueous component that can be in the form of a lotion, a cream, a gel, or a paste. Examples of suitable liposomal bases include PCCA Lipoderm®, Lipoderm ActiveMax™, Anhydrous Lipoderm, and Lipoderm High Molecular Weight™PCCA. Such liposomal base formulations can include, for example, about 60-80% wt/wt water combined with glycerin, C₁₂₋₁₅ alkyl benzoate, glyceryl stearate, dimethicone, cetearyl alcohol, cetearyl glucoside, polyacrylamide, cetyl alcohol, magnesium aluminum silicate, xanthan gum, aloe vera (aloe barbadensis), tocopheryl acetate (vitamin E acetate), prunus amygadalus amara (bitter almond) kernel oil, vitis vinifera (Grape) seed extract, triticum vulgare (wheat) germ oil, retinyl palmitate (vitamin A palmitate), ascorbyl palmitate (vitamin C palmitate), Pro-Lipo Multi-emulsion Liposomic System, tetrasodium EDTA, phenoxyethanol, sodium hydroxymethylglycinate and the like and combinations thereof.

In some embodiments, the base may be cream base. Cream bases are semi-solid emulsions of oil and water. They are divided into two types: oil-in-water (O/W) creams which are composed of small droplets of oil dispersed in a continuous water phase, and water-in-oil (W/O) creams which are composed of small droplets of water dispersed in a continuous oily phase. Oil-in-water creams are more comfortable and cosmetically acceptable as they are less greasy and more easily washed off using water. Water-in-oil creams are more difficult to handle but many drugs which are incorporated into creams are hydrophobic and will be released more readily from a water-in-oil cream than an oil-in-water cream. Water-in-oil creams are also more moisturising as they provide an oily barrier which reduces water loss from the stratum corneum, the outermost layer of the skin. Cream bases typically include water, oil, emulsifier, and thickening agents, such as those discussed below.

In some embodiments, the base may be a moisturizing cream base. Moisturizing cream bases are composed of the same components as the cream bases described above with the addition of an emollient or humectant, that may provide a barrier that reduces water loss from the stratum corneum, the outermost layer of the skin. The emollient or humectant in a moisturizing cream base may be cetyl esters wax, stearyl alcohol, cetyl alcohol, and glycerin, or combinations thereof.

Example cream bases and moisturizing cream bases include VersaBase (PCCA); Emollient cream, Vanishing cream, CeraVe, Vanicream, Vitamin E; Cliniderm; Dermabase (purified water, petrolatum, mineral oil, cetostearyl alcohol); Eucerin (water, petrolatum, mineral oil, ceresin, lanolin alcohol, methylchloroisothiozolinone, methylisothiazolinone); Glaxal (WellSpring Pharmaceutical Corp., Sarasota, Fla.); stearic acid cream, or any other pharmaceutical cream base used for topical formulations known to those skilled in the art.

In some embodiments, the base may be an ointment base. Ointments are compositions in which oil and water are provided in a ratio of from 7:1 to 2:1, from 5:1 to 3:1, or 4:1, and in some embodiments, the ointment may or may not include water, such as Aquaphor, Pracasil, and plasticized bases. Ointments are generally formulated using oils, waxes, water, alcohols, petroleum products, silicones, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

The amount of base in the compositions of embodiments can vary and will depend on the amounts of the other components. More base can be added to compensate for smaller amounts of other components in the desired topical pharmaceutical formulation. In some embodiments, the base may be present in a concentration of about 45% (w/w) to about 99.75% (w/w) of the total composition, or any range or individual concentration known in the art.

The compositions of various embodiments affect treatment of the various skin diseases including, for example, epidermolysis bullosa simplex, congenital aplasia cutis, neonatal pemphigus, neonatal herpes gestationis, staphylococcal scalded skin syndrome, incontinentia pigmenti, epidermolytic ichthyosis, linear IgA dermatosis, bullous pemphigoid, bullous impetigo, bullous pemphigoid, bullous impetigo, bullous lichen planus, lichen planus of the mucosa, tuberous sclerosis-associated angiofibromas, angiofibromas, trichoepitheliomas, skin lesions associated with Birt-Hogg-Dube syndrome, of the skin of the face, skin lesions associated with Langerhans Cell Histiocytosis, Vascular malformations and tumors, Port Wine Stains, Kaposi sarcoma, Epidermal nevi, treatment-resistant hemangiomas, sensitive skin, fragile skin, or combinations thereof. However, certain formulations are more effective for treating particular skin diseases. For example, a composition containing up to about 2%, up to about 5%, or up to about 15% JAK/STAT inhibitor in a non-comedogenic, hypoallergenic, unscented, cosmetic cream base that is easily absorbed by the skin may well-suited for treating tuberous sclerosis-associated angiofibromas, angiofibromas, trichoepitheliomas, skin lesions associated with Birt-Hogg-Dube syndrome, other overgrowths related to the skin of the face, skin lesions associated with Langerhans Cell Histiocytosis, cutaneous lupus erythematosus, icthyosis, neurofibromas, and the like. In other embodiments, a composition containing up to about 2%, up to about 5%, or up to about 15% JAK/STAT inhibitor in a liposomal cream base may be well-suited to treat deeper lesions such as Vascular anomalies (malformations and tumors), Port Wine Stains, Kaposi sarcoma, Epidermal nevi, treatment-resistant hemangiomas, cutaneous leiomyomas, acanthosis nigricans, confluent and reticulated papillomatosis, neurofibromas, neurofibromas associated with neurofibromatosis, and the like. In further embodiments, a composition containing up to about 2%, up to about 5%, or up to about 15% JAK/STAT inhibitor in a water-free ointment base may be well-suited to treat, bullous pemphigoid, bullous impetigo, bullous lichen planus, lichen planus of the mucosa, and the like or any condition in patients with extremely sensitive or fragile skin. In still other embodiments, composition containing up to about 2%, up to about 5%, or up to about 15% JAK/STAT inhibitor in a moisturizing cream base can be used to treat any of the conditions identified above in patients with sensitive skin, including newborns and infants.

The compositions of various embodiments can be in other forms, including lotions, foams, liniments, balms, soaps, shampoos, mouth wash, and the like.

In some embodiments, the topical formulations can be in the form of a lotion. Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

In some embodiments, the topical formulations can be in the form of a foam. Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Accordingly, this delivery technology is a useful addition to the spectrum of formulations available for topical use.

In some embodiments, the topical formulations can be in the form of a liniment. Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicylate, benzoin resin, or capsaicin.

In some embodiments, the formulations can be in the form of a soap, which are formulations that comprise a salt of a fatty acid. Soaps are mainly used as surfactants for washing, bathing, and cleaning, but they are also used in textile spinning and are important components of lubricants. Soaps for cleansing are usually obtained by treating vegetable or animal oils and fats with a strongly alkaline solution. Fats and oils are composed of triglycerides; three molecules of fatty acids are attached to a single molecule of glycerol. The alkaline solution, which is often called lye (although the term “lye soap” refers almost exclusively to soaps made with sodium hydroxide), is believed to promote a chemical reaction known as saponification. In saponification, the fats are first hydrolyzed into free fatty acids, which then combine with the alkali to form crude soap. Glycerol (glycerin) is usually liberated and is either left in or washed out and recovered as a useful byproduct, depending on the process employed.

In some embodiments, the topical formulations can be in the form of a shampoo, which is a hair care product used for the removal of oils, dirt, skin particles, dandruff, environmental pollutants, and other contaminant particles that gradually build up in hair. A goal may be to remove the unwanted build-up without stripping out so much sebum as to make hair unmanageable.

In some embodiments, the topical formulations can be in the form of a suppository. Suppository formulations can be prepared by admixing a therapeutically effective amount of JAK/STAT inhibitor as discussed above with a suppository base and forming suppositories from the admixture by any art recognized method of making suppositories. The suppository base is typically lipophilic and, in some embodiments, can be an aprotic lipophilic base such as a triglyceride lipophilic base or a paraffinic base comprising mixtures of hydrocarbons. The suppository base may have a melting temperature of from about 32° C. to 36° C. or a triglyceride mixture of fatty acids having a melting point range of from about 32° C. to 36° C. The mixture of hydrocarbons can preferably be a mixture of hard paraffin (about 50-60%) and liquid paraffin (about 40-50%) having a melting point range of about 32° C. to 36° C.

In some embodiments, the compositions may be formulated as a mouthwash, oral rinse, or oral spray. Mouthwash and oral rinses are well known in the dental arts and are liquid preparations that are specifically designed to cleanse the mouth. Mouthwashes, rinses, gargles and sprays generally include water, ethanol, humectant, and in some embodiments, surfactant, flavoring agents, sweetening agents, coloring agent, and the like and combinations thereof. In some embodiments, such compositions may include a thickening agent, one or more anticaries agents, anticalculus agents and the like and combinations thereof. A typical composition contains about 0% to about 80% of a humectant, about 0.01% to about 7% of a surfactant, about 0.03% to about 2% of a flavoring agent, about 0.005% to about 3% of a sweetening agent, about 0.001% to about 0.5% of a coloring agent, with the balance being water. Another typical composition contains about 5% to about 60% or about 5% to about 20% ethanol, about 0% to about 30% or about 5% to about 20% humectant, about 0% to about 2% emulsifying agents or about 0% to about 0.5% of a sweetening agent, about 0% to about 0.3% of a flavoring agent, and water. Such compositions may also include about 0.05% to about 0.3% of an anticaries agent, about 0.1% to about 3% of an anticalculus agent, or combinations thereof.

In certain embodiments, the suppository base may be a solid adjuvant mixture that is about 80% to about 90% by weight water-soluble, and in some embodiments, the suppository base may include solid polyethylene glycol, a liquid polyethylene glycol that is soluble in the solid polyethylene glycol, solid oil-soluble surfactant, a water-soluble surfactant, and spermaceti. The physical properties of the various individual ingredients, by interaction, contribute to the properties of the formulated composition the characteristics which guarantee extrudability, water-dispersibility, and storage-stability. The amounts and proportions of the various ingredients of the base will vary with the amounts of the medicinal ingredients incorporated therein. In some embodiments, the solid polyethylene glycol may be about 23% to about 35% by weight of the total composition and the liquid polyethylene glycol may be about 10% to about 13% by weight of the total composition. The solid polyethylene glycol may have a molecular weight of about 4000 to about 6000, and the liquid polyethylene glycol may have a molecular weight of about 200 to about 600. The solid oil-soluble surfactant may be about 9% to about 11% by weight of the total composition and may be polyoxyethylene sorbitan monostearate (Tween 61) or polyoxyethylene sorbitan tristearate (Tween 65). The water-soluble surfactant may be about 4% to about 12% by weight of the total composition and can be an ethylene oxide-polyproplyene gylcol condensation product. Spermaceti can be about 26% to about 40% by weight of the total composition. A solid adjuvant can be beta lactose, sucrose, dextrose, sodium chloride, sodium sulfate, and the like and combinations thereof, and in some embodiments, the suppository formulation may include a starch such as corn starch, which can be mixed with small amounts of methylcellulose, guar gum, or purified wood cellulose.

Example compositions may include various known components. For example, in some embodiments, the composition may include a solvent such as isopropyl alcohol, benzyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), Transcutol, propylene glycol (PG), N-methyl-2pyrrolidone (NMP), methylene chloride, diethyl ether, ethanol, acetonitrile, ethyl acetate, a combination of natural oil; ethylene glycol, propylene glycol, dimethyl polysiloxane (DMPX), oleic acid, caprylic acid, 1-octanol, ethanol (denatured or anhydrous), liposomal compositions, suitable plant oils, such as Aloe vera derivatives or sesame seed oil or derivatives thereof, acrylic polymers, rubber-based polymers, polysiloxane-based polymers, polyvinylpyrrolidone-based polymers, dimethylsulfoxide (DMSO), dimethylformamide (DMF), dimethylacetamide, N-methyl-2-pyrrolidone, hexamethylphosphoramide (HMPA), lecithin, Transfersomes® (bi-component vesicular aggregates), ethosomes, azone, castor oil derivatives, such as ethoxylated castor oil, jojoba oil derivatives, corn oil derivatives, emu oil derivatives, and the like and combinations thereof. The solvent can be present in a concentration of about 5.0% (w/w) to about 15.0% (w/w)., about 6.0% (w/w) to about 10.0% (w/w), about 7.5% (w/w) to about 10.5% (w/w), about 8.0% (w/w) to about 10.0% (w/w), or any range or individual concentration of solvent encompassed by these example ranges.

In some embodiments, the compositions may include an antioxidant. Such antioxidant may be, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecyl sulphate, sodium lauryl sulphate (SLS), a polyoxyethylene ester of polysorbitan, such as monooleate, monolaurate, monopalmitate, monostearate esters, esters of sorbitan, the polyoxyethylene ethers, the sodium dioctyl sulfosuccinate (DOSS), lecithin, and sodium docusate. The amount of humectant in such compositions may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphone, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

In some embodiments, the compositions may further include a topical debriding agent such as, for example, papain/urea, balsam peru/castor oil/trypsin, chlorophyllin copper complex/papain/urea, collagenase, and the like and combinations thereof. The amount of the debriding agent in such compositions may be about 0.01% (w/w) to 5% (w/w) of the total composition.

In some embodiments, the compositions may further include a topical emollient such as, for example, urea, ammonium lactate, salicylic acid/urea, vitamins A, D, and E, ammonium lactate/pramoxine, vitamin A & D, dexpanthenol, ammonium lactate/urea, salicylic acid/urea, aloe vera, lanolin, and the like and combinations thereof. The amount of the emollient such compositions may be about 0.01% (w/w) to 5% (w/w) of the total composition.

A cream base may be prepared by conventional techniques well known to those skilled in the art. Generally, a suitable process includes admixing the various ingredients of the cream in appropriate relative amounts in any order that is convenient and thereafter, if necessary adjusting the pH to the final desired value. For example, the components of the base may be mixed together at a temperature of about 65° C. to about 75° C. until an emulsion has formed, and therapeutic agent may be added after cooling the emulsified cream base or during mixing.

Other embodiments of the invention include methods for treating skin diseases and skin lesions by administering the compositions described above. The methods of various embodiments may include the steps of administering a composition of the various embodiments described above to the location of skin disease or skin lesion of the subject in need of treatment. For example, the step of administering can include applying the compositions of embodiments to the skin of a patient in need of treatment. The step of administering can be carried out by various means. For example, administering can be accomplished by applying the composition to the skin of an infected subject, and in some embodiments, the skin may be massaged or rubbed to facilitate contacting the affected area. In some embodiments, the step of administering can be carried out one, two, three, four, or more times per day, and administering can be carried out the prescribed number of times per day for one week to six months or until the symptoms are resolved. In some embodiments, improvement in one or more symptoms may be observed within about 7 days of treatment, and in certain embodiments, improvement in one or more symptoms may be observed within about 1, about 2, about 3, about 4, about 5, or about 6 days after initial treatment.

As is known in the art, certain means for administering may require the use of particular components of the formulation. Such components are described above and can be appropriately incorporated into the compositions.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

TABLE 1
Example compositions are provided in TABLE 1.

| | Ex. 1 | Ex. 2 | Ex. 3 | Ex. 4 | Ex. 5 | Ex. 6 | Ex. 7 | Ex. 8 | Ex. 9 | Ex. 10 | Ex. 11 | Ex. 12 | Ex.13 | Ex. 14 |
|---|---|---|---|---|---|---|---|---|---|---|---|---|---|---|
| Tofacitinib | 1% | 1% | 1% | 1% | 1% | 1% | 1% | 1% | 1% | 1% | 1% | 1% | 1% | 1% |
| Benzyl | 2.5% | | 2.5% | | 2.5% | | 2.5% | | 2.5% | | 2.5% | | 2.5% | |
| Alcohol | | | | | | | | | | | | | | |
| Versabase | 96.5% | 99% | | | | | | | | | | | | |
| Cream* | | | | | | | | | | | | | | |
| Lipoderm | | | 96.5% | 99% | | | | | | | | | | |
| Pracasil | | | | | 96.5 | 99% | | | | | | | | |
| Petrolatum | | | | | | | 96.5% | 99% | | | | | | |
| Plasticized | | | | | | | | | 96.5% | 99% | | | | |
| Base | | | | | | | | | | | | | | |
| Emollient | | | | | | | | | | | 96.5% | 99% | | |
| Cream | | | | | | | | | | | | | | |
| Aquaphor | | | | | | | | | | | | | 96.5% | 99% |

*versabase cream, vanishing cream, CeraVe, or VaniCream

1. A method for treating skin conditions, comprising topically administering to a subject in need of treatment a composition comprising up to about 5% (w/w) of a JAK/STAT inhibitor and a base.

2. The method of claim 1, wherein the composition is in the form of a lotion, foam, liniment, balm, soap, shampoo, suppository and the like and combinations thereof.

3. The method of claim 1, wherein the JAK/STAT inhibitor is selected from the group consisting of ruxolitinib (INCB 018424), tofacitinib (CP690550), AG490, momelotinib (CYT387), partcitinib (SB 1518), baricitinib (LY3009104), fedratinib (TG101348), BMS-911543, lestaurtinib (CEP- 701), fludarabine, epigallocatechin-3-gallate (EGCG), baricitinib, momelotinib, pacritinib, peficitinib, ABT 494, AT 9283, decernmotinib, filgotinib, gandotinib, INCB 39110, PF 4965842, R348, AZD 1480, BMS 911543, cerdulatinib, INCB 052793, NS 018, C 410, CT 1578, JTE 052, PF 6263276, R 548, TG 02, lumbricus rebellus extract, ARN 4079, AR 13154, UR 67767, CS510, VR588, DNX 04042, hyperforin, and pharmaceutically acceptable salts and combinations thereof.

4. The method of claim 1, wherein JAK/STAT inhibitor is tofacitinib.

5. The method of claim 1, wherein the base is selected from the group consisting of white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, versabase, and combinations thereof.

6. The method of claim 1, wherein the base has a concentration of about 45% (w/w) to about 99.75% (w/w) of the total composition.

7. The method of claim 1, wherein the skin condition is selected from the group consisting of Epidermolysis bullosa simplex, congenital aplasia cutis, neonatal pemphigus, neonatal herpes gestationis, staphylococcal scalded skin syndrome, incontinentia pigmenti, epidermolytic ichthyosis, linear IgA dermatosis, bullous pemphigoid, bullous impetigo, bullous lichen planus, lichen planus of the mucosa, tuberous sclerosis-associated angiofibromas, angiofibromas, trichoepitheliomas, skin lesions associated with Birt-Hogg-Dube syndrome, of the skin of the face, skin lesions associated with Langerhans Cell Histiocytosis, Vascular malformations and tumors, Port Wine Stains, Kaposi sarcoma, Epidermal nevi, treatment-resistant hemangiomas, sensitive skin, fragile skin, or combinations thereof.

8. The method of claim 1, wherein the composition further comprises a solvent, antioxidant, emulsifying agent, humectant, analgesic agent, topical debriding agent, topical emollient, and the like and combinations thereof.

9. The method of claim 1, wherein the composition further comprises a solvent.

10. The method of claim 11, wherein the solvent is selected from the group consisting of isopropyl alcohol, benzyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), Transcutol, propylene glycol (PG), N-methyl-2 pyrrolidone (NMP), methylene chloride, diethyl ether, ethanol, acetonitrile, ethyl acetate, ethylene glycol, propylene glycol, dimethyl polysiloxane (DMPX), oleic acid, caprylic acid, 1-octanol, ethanol (denatured or anhydrous), and combinations thereof.

11. The method of claim 11, wherein the solvent has a concentration of about 5.0% (w/w) to about 15.0% (w/w).

12. The method of claim 1, wherein the composition further comprises an antioxidant selected from the group consisting of butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and pharmaceutically acceptable salt and ester thereof, and combinations thereof.

13. The method of claim 14, wherein the antioxidant has a concentration of about 0.01% (w/w) to about 1% (w/w).
